(19) United States
(12) Patent Application Publication
(10) Pub. No.: US 2006/0121807 A1
(43) Pub. Date: Jun. 8, 2006

(54) Soap-containing cleansing substrate

(76) Inventors: Harald Albrecht, Hamburg (DE); Albrecht Dorschner, Hamburg (DE); Jorg Kuther, Halstenbek (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/523,440

(22) PCT Filed: Jul. 16, 2003
(86) PCT No.: PCT/EP03/07728

§ 371 Date: Oct. 7, 2005

(30) Foreign Application Priority Data
Jul. 27, 2002 (DE) 10234260.1

(51) Int. Cl. B32B 27/04; B32B 27/12; B32B 5/02; A61K 8/02

(52) U.S. Cl. 442/121; 442/123; 424/401

(57) ABSTRACT

Substrate impregnated with a cosmetic preparation containing one or more fatty acid salts.

The present invention relates to a cleansing substrate impregnated with a cosmetic preparation containing fatty acid salts, the production and use thereof.

The desire for clean skin is probably as old as mankind, because dirt, perspiration, and residues of dead skin particles offer an ideal breeding ground for all kinds of pathogens and parasites. Probably, the oldest preparations for cleansing the skin are soaps, whose formulations had been recorded on clay tablets from Mesopotamia as early as 2500 B.C. Although the use and consumption of soaps has been declining since the development of cleansing surfactants, they continue to take a firm position in the variety of cleansing preparations, last but not least because of their low production costs.

Soaps develop during the reaction (“saponification”) of a fat or fatty acids or fatty acid methyl esters obtained therefrom with sodium or potassium hydroxide solution. From the viewpoint of chemistry, they represent the alkali salt of fatty acids.

As neutral fats it is common to use beef tallow, palm oil, palm kernel oil, or coconut oil. Important for the property of the soap is the distribution of the chain lengths of corresponding fatty acids. Thus, a high concentration of laurate soap (produced from lauric acid=dodecanoic acid) results in a particularly well foaming soap. Likewise, the selection of the cation needed for forming the salt has an influence on the properties of the soap. Sodium soaps are in most cases solid at room temperature, whereas potassium soaps normally have a soft and pasty consistency [W. Umbach (Hrsg: Kosmetik, Entwicklung, Herstellung und Anwendung kosmetischer Mittel, 2^{nd} Ed., Thieme Verlag, Stuttgart, 1995].

A special product form for cleansing preparations are solid cleansing substrates, in particular wipes. These can be impregnated already by the manufacturer with the cleansing preparation, and thus have the advantage that they already contain the preparation in the correct dosage. Furthermore, they avoid the disadvantage of preparations stored in bottles, whose packaging may break and whose contents may “leak out.” Further advantages of cleansing substrates/wipes also include the situation that they are easy to take along on trips in counted quantities, and that they normally require no longer water for their application.

Cleansing substrates/wipes are made from textiles. The textiles may be woven or knit, and be present as composite material (nonwoven textile). In most cases, composite materials are used (for cost reasons). In the case of composite materials, the fabric is not produced by warp and weft or stitch formation, but by interlacing, and/or cohesive, and/or adhesive bonding of textile fibers. According to DIN 61210 T2, composite materials may be distinguished by nonwoven webs, paper, batting, and felt. Nonwoven webs are loose materials produced from spun fibers (i.e. fibers of a defined length), or filaments (endless fibers), in most cases of polypropylene, polyester, or rayon. Typically, their cohesion is provided by the fibers holding together. In this connection, the individual fibers may have a preferred orientation (oriented or cross-laid webs), or be unoriented (random webs). The nonwovens may be mechanically bonded by needle punching, stitching, or entangling by means of strong water jets. Adhesively bonded nonwovens are produced by gluing the fibers together with liquid binding agents (for example, acrylate polymers, SBR/NBR, polyvinyl ester, polyurethane dispersions), or by melting or dissolving so-called binder fibers that are added to the web during its production. In the case of cohesive bonding, the fiber surfaces are partially dissolved by suitable chemicals and bonded by pressure or fused at an increased temperature [J. Falbe, M. Regnitz: Römpp-Chemie-Lexikon, 9^{th} edition, Thieme-Verlag, Stuttgart (1992)].

Substrates that are impregnated with cosmetic preparations and in particular wipes may be produced in different ways: in a so-called “dip method”, the wipe is immersed into a dip bath or pulled through a bath. This method is especially suited for paper wipes and less suited for nonwoven fabrics, since the latter absorb too much liquid (=preparation), and when being repackaged, subsequently find themselves in puddles of the preparation that has been released again.

A second variant is the “spray method”, wherein the preparation is sprayed onto the advancing fabric. While this method is suitable for all textiles, it does not permit applying heavily foaming preparations to the fabric, since the foam development becomes too great in the spray method.

As further methods, so-called squeegee methods are used, wherein webs of nonwovens or wipes advance along doctor blades, doctor bars, or doctor nozzles, which continuously receive an impregnation solution. Different degrees of impregnation may be adjusted, among other things, by varying the contact pressure and the rate of advance of the fabric.

In the art, it has so far been possible only to a satisfactory extent to apply heavily foaming preparations to paper wipes by the “dip method.” Heavily foaming preparations are primarily used as cleansing preparations, because foam greatly increases their cleansing efficiency. The disadvantage of paper wipes vis-à-vis nonwovens is their low breaking strength. Likewise, nonwovens are substantially softer on the skin and thus more protective. Nonwovens are especially suited for cleansing the face. Last but not least nonwovens are clearly whiter than paper wipes, which makes on consumers a clearly more hygienic impression.

Disadvantageous in the art is also the situation that the cleansing substrates must be dried after impregnating them with the cleansing preparation, when they are intended to be presented to the user in the form of so-called “dry wipes.” This drying process is especially time and energy consuming, and thus represents a decisive cost factor in the production of the wipes. Moreover, in the case of surfactant-containing cleansing preparations, the preparation must contain very high concentrations of surfactants, so that the end product exhibits in use adequately large amounts of foam, a high cleansing efficiency, as well as a pleasant creaminess of the foam. High surfactant concentrations, however, may also lead to problems with the formulation of the preparation and slow down the drying process.

It was therefore an object of the present invention to develop substrates, which do not have the disadvantages of the state of the art, or have them only to a small extent.

Surprisingly, the object is accomplished by a substrate that is impregnated with a cosmetic preparation containing one or more fatty acid salts.

The substrates of the invention represent cleansing utensils, which exhibit a high cleansing efficiency when being used, and which distinguish themselves by an excellent foaming ability (large amount of foam), as well as a pleasant creaminess of the foam.

Impregnated substrates mean in accordance with invention substrates, which have been impregnated by spraying, dipping, or spreading thereon the cosmetic preparation. The term impregnated comprises both freshly impregnated substrates with a moist touch and substrates which have been dried by a drying process or have a dry touch, and which contain the cosmetic preparation in a concentrated form.

The substrates of the invention may be smooth or also surface-structured. According to the invention, surface-structured substrates are preferred.

In the case of the substrates according to the invention, the fabric can be produced by warp or weft, by stitch formation or by interlacing, and/or cohesive, and/or adhesive bonding of textile fibers. In accordance with the invention, the substrate is preferably a composite material.

According to the invention, it is preferred to use substrates in the form of wipes, which consist of a nonwoven fabric, in particular of water-jet bonded and/or water-jet embossed nonwovens. Advantageously, the substrates may also be realized as bulk material, perforated nonwoven, or mesh fabric.

Substrates of this type may have macro-embossed surfaces of any desired pattern. The selection to be made depends on the one hand on the impregnation being applied, and on the other hand on the field of application, in which the subsequent wipe is to be used.

If embossed nonwovens are used, large cavities on the surface of the nonwoven and in the nonwoven will facilitate absorption of dirt and impurities, when the impregnated wipe is moved over the skin. In comparison with smooth wipes, it is possible to increase the cleansing effect by a multiple.

It has been found advantageous for the wipe, when same has a weight of 20 to 120 g/m², preferably 30 to 80 g/m², more preferably 40 to 60 g/m² (measured at 20° C.+2° C. and at a humidity of the room air of 65%±5% for 24 hours).

The thickness of the nonwoven is preferably from 0.2 mm to 2 mm, in particular 0.4 mm to 1.5 mm, preferably 0.6 mm to 0.9 mm.

As basic materials for the nonwoven fabric of the wipe, it is possible to use in general all organic and inorganic fiber materials on natural and synthetic basis. Examples include cellulose, jute, hemp, sisal, silk, wool, polypropylene, polyethylene therephthalate (PET), aramid, nylon, polyvinyl derivatives, polyurethane, polylactide, polyhydroxy alkanoate, cellulose ester and/or polyethylene, as well as mineral fibers, such as glass fibers or carbon fibers. The present invention, however, is not limited to the referenced materials. It is also possible to use a plurality of further fibers for constructing the nonwoven. It is especially advantageous for the purposes of the present invention when the used fibers are water-insoluble.

In an advantageous embodiment of the nonwoven, the fibers consist of a blend of 60% to 80% rayon, with 40% to 20% PET, in particular 70% rayon and 30% PET. Especially advantageous is a blend of 70% viscose and 30% PET.

Especially advantageous are also fibers of high-tenacity polymers, such as polyamide, polyester and/or highly oriented polyethylene.

Moreover, the fibers may also be dyed to be able to highlight and/or enhance the optical attractiveness of the nonwoven. The fibers may contain in addition UV stabilizers and/or preservatives.

The fibers used for producing the fabric preferably have a water absorption rate of more than 60 mm/[10 min.] (measured by the EDANA test 10.1-72), in particular more than 80 mm/[10 min.].

Furthermore, the fibers used for producing the fabric preferably have a water absorption capacity of more than 5 g/g (measured by the EDANA test 10.1-72), in particular more than 8 g/g.

Advantageous fabrics for the purposes of the present invention have a tensile strength, in particular

| | | [N/50 mm] |
|---|---|---|
| in the dry state | machine direction | >60, preferably >80 |
| | cross direction | >20, preferably >30 |
| in the impregnated | machine direction | >4, preferably >60 |
| state | cross direction | >10, preferably >20 |

The elongation of the advantageous fabric is preferably

| | | |
|---|---|---|
| in the dry state | machine direction | 15% to 100%, |
| preferably | | 20% and 50% |
| | cross direction | 40% to 120%, |
| preferably | | 50% and 85% |
| in the impregnated | machine direction | 15% to 100%, |
| state | | |
| preferably | | 20% and 40% |
| | cross direction | 40% to 120%, |
| preferably | | 50% and 85% |

In accordance with the invention, the weight ratio of nonwoven to cleansing substrate is preferably in a range from 1:0.5 to 1:5. Especially preferred is a range from 1:0.7 to 1:3.

In accordance with the invention, the advantageous fatty acid salts are selected from the group of the fatty acids which comprise 8 to 20 carbon atoms per molecule.

Preferred fatty acids in accordance with the invention include lauric acid (dodecanoic acid), myristic acid (tetradecanoic acid), palmitic acid (hexadecanoic acid), stearic aid (octadecanoic acid).

In accordance with the invention, it is likewise preferred to use the sodium and potassium salts of the fatty acids according to the invention.

It is especially preferred for the purposes of the present invention to select the fatty acid salt or salts from the group of potassium laurate, potassium myristate, potassium palmitate, potassium stearate, and potassium oleate.

It is advantageous in accordance with the invention, when the cosmetic preparation contains at the time of its application to the substrate one or more fatty acid salts in a concentration from 0.1 to 20% by weight, preferably 1 to 15% by weight, and more preferably 2 to 20% by weight based on the total weight of the preparation.

In accordance with the invention it is also of advantage, when the cosmetic cleansing preparation contains besides one or more fatty acid salts further cosmetic ingredients, auxiliaries, and/or additives.

For example, it is advantageous in accordance with the invention, when the cosmetic preparation contains one or more surfactants.

It is especially advantageous to select the wash-active surfactant or surfactants of the invention from the group of surfactants, which have an HLB value greater than 25. Most advantageous are those, which have an HLB value greater than 35.

In accordance with the invention advantageous anionic surfactants for the purposes of the present invention are:

Acylamino acids and salts thereof, such as
acyl glutamates, in particular sodium acyl glutamate
sarcosinates, for example, myristoyl sarcosinate, TEA-lauroyl sarcosinate, sodium lauroyl sarcosinate, and sodium cocoyl sarcosinate;

Sulfonic acids and salts thereof, such as
acyl isethionates, for example, sodium/ammonium cocoyl isethionate,
sulfosuccinates, for example, dioctyl sodium sulfosuccinate, disodium laureth sulfosuccinate, disodium lauryl sulfosuccinate, and disodium undecylenamido MEA sulfosuccinate; as well as sulfuric esters, such as
alkyl ethers sulfates, for example, sodium-, ammonium-, magnesium-, MIPA-, TIPA-laureth sulfate, sodium myreth sulphate, and sodium C₁₂₋₁₃ pareth sulfate,
alkyl sulfates, for example sodium-, ammonium-, and TEA lauryl sulfate.

In accordance with the invention, it is also possible and advantageous to use
taurates, for example, sodium lauroyl taurate and sodium methyl cocoyl taurate,
ether carboxylic acids, for example, sodium laureth-13 carboxylate and sodium PEG-6 cocamide carboxylate, sodium PEG-7 olive oil carboxylate,
phosphoric acid esters and salts, such as, for example, DEA oleth-10 phosphate and dilaureth-4 phosphate,
alkylsulphonates, for example sodium cocomonoglyceride sulfate, sodium C₁₂₋₁₄ olefin sulfonate, sodium lauryl sulfoacetate, and magnesium PEG-3 cocoamide sulfate.

In accordance with the invention it is preferred to use as anionic surfactants acylamino acid surfactants, sarcosinates, sulfosuccinates, sulfosuccinate citrates, monoalkyl phosphates, and olefin sulfonates.

In accordance with the invention, it is especially preferred to use as anionic surfactants acylamino acid surfactants, such as sodium cocoylglutamate (for example, Aminosoft CS-11 from Ajinomoto), sodiumlauroyl glutamate, sodium capryloyl glutamate, and/or Di-TEA-palmitoyl aspartate.

Especially advantageous, wash-active cationic surfactants for the purposes of the present invention are quaternary surfactants. Quaternary surfactants contain at least one N-atom, which is covalently bonded to 4 alkyl or aryl groups. Advantageous are benzalkonium chloride, alkyl betaine, alkylamidopropyl betaine, and alkylamidopropyl hydroxysultaine.

Especially advantageous wash-active amphoteric surfactants for the purposes of the present invention are
acyl/dialkylethylenediamine, for example, sodium acyl amphoacetate, disodium acyl amphodipropionate, disodium alkyl amphodiacetate, sodium acyl amphohydroxypropyl sulfonate, disodium acyl amphodiacetate, and sodium acyl amphopropionate.

Especially advantageous wash-active nonionic surfactants for the purposes of the present invention are
alkanolamides, such as cocamides MEA/DEA/MIPA,
esters, which are formed by esterification of carboxylic acids with ethylene oxide, glycerol, sorbitan, or other alcohols,
ethers, for example ethoxylated alcohols, ethoxylated lanolin, ethoxylated polysiloxanes, propoxylated POE ethers, and alkylpolyglycosides, such as laurylglucoside, decylglycoside, and cocoglycoside.

Further advantageous anionic surfactants are
taurates, for example, sodium lauroyl taurate and sodium methylcocoyl taurate,
ether carboxylic acids, for example, sodium laureth-13 carboxylate, and sodium PEG-6 cocoamide carboxylate, sodium PEG-7 olive oil carboxylate,
esters of phosphoric acid and salts, such as, for example, DEA-oleth-10-phosphate and dilaureth-4 phosphate,
alkyl sulfonates, for example, sodium cocomonoglyceride sulfate, sodium C₁₂₋₁₄ olefin sulfonate, sodium lauryl sulfoacetate, and magnesium PEG-3 cocamide sulfate.

Additional advantageous amphoteric surfactants are:
N-alkylamino acids, for example, aminopropylalkyl glutamide, alkylaminopropionic acid, sodium alkylimidodipropionate, and lauroamphocarboxyglycinate.

Additional advantageous nonionic surfactants are alcohols.

Further suitable anionic surfactants for the purposes of the present invention also include
acylglutamates, such as Di-TEA-palmitoylaspartate and sodium caprylic/capric glutamate,
acylpeptides, for example palmitoyl-hydrolized milk protein, sodium cocoyl-hydrolized soy protein, and sodium-/potassium cocoyl-hydrolized collagen,
   as well as carboxylic acids and derivatives, such as
for example, lauric acid, aluminum stearate, magnesium alkanolate, and zinc undecylenate,
ester-carboxylic acids, for example, calciumstearoyllactylate, laureth-6 citrate, and sodium PEG-4 lauramide carboxylate,
alkylaryl sulfonates.

Further suitable cationic surfactants for the purposes of the present invention also include
alkylamines,
alkylimidazoles,
ethoxylated amines.

Further suitable nonionic surfactants for the purposes of the present invention also include amine oxides, such as cocoamidopropylamine oxide.

It is advantageous for the purposes of the invention to select the content of one or more of the wash-active surfactants in the cosmetic preparation in a range from 0.1 to 25% by weight, preferably 1 to 15% by weight, and more preferably 2 to 10% by weight, each based on the total weight of the preparation.

In accordance with the invention, the preparations of the invention may advantageously contain polysorbates. Polysorbates are a class of compounds that derive from sorbitan, a furan derivative that is obtained from sorbitol by the separation of two equivalents of water. The hydroxyl groups of the sorbitan are etherified with polyethylene glycols, whose ends may be esterified with fatty acids. They can be generally represented by the formula 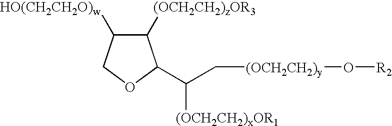
where R₁, R₂, R₃═H, fatty acid residue.

For the purposes of the invention, advantageous polysorbates are, for example,
Polyoxyethylene(20)sorbitanmonolaurate (Tween 20, CAS-No. 9005-64-5)
Polyoxyethylene(4)sorbitanmonolaurate (Tween 21, CAS-No. 9005-64-5)
Polyoxyethylene(4)sorbitanmonostearate (Tween 61, CAS-No. 9005-67-8)
Polyoxyethylene(20)sorbitantristearate (Tween 65, CAS-No. 9005-71-4)
Polyoxyethylene(20)sorbitanmonooleate (Tween 80, CAS-No. 9005-65-6)
Polyoxyethylene(5)sorbitanmonooleate (Tween 81, CAS-No. 9005-65-5)
Polyoxyethylene(20)sorbitantrioleate (Tween 85, CAS-No. 9005-70-3).

In accordance with the invention, these polysorbates are advantageously used individually or as combination of several polysorbates in a concentration from 0.1 to 5% by weight, and in particular in a concentration from 1.5 to 2.5% by weight based on the total weight of the preparation.

The substrates of the invention contain preservatives in the cleansing preparation with which they are impregnated. Advantageous preservatives for the purposes of the invention are, for example, formaldehyde donors (such as, for example, DMDM hydantoin, which is available under the trade name Glydant™ from Lonza), iodopropyl butylcarbamates (for example, those which are available under the trade names Glycacil-S from Lonza and/or Dekaben LMB from Jan Dekker), parabens (i.e., alkyl esters of the p-hydroxybenzoic acid, such as methyl-, ethyl-, propyl-, and/or butylparaben), phenoxyethanol, ethanol, benzoic acid, and the like. Normally, the preservation system furthermore comprises in accordance with the invention advantageously preservative auxiliaries, such as, for example octoxyglycerin, glycine soya, etc. The following table gives an overview of some preservatives that are advantageous in accordance with the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na salt |
| E 218 | Methyl p-hydroxybenzoate |
| E 219 | Methyl p-hydroxybenzoate Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrate |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also advantageous according to the invention are preservatives or preservative auxiliaries that are customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinyl urea, 5-chloro-2-methyl-4-isothiazolin-3-one, 2-chloroacetamide, benzalkonium chloride, and benzyl alcohol.

According to the invention, it is especially advantageous to use as preservatives benzoic acid, and/or salicylic acid, and/or their derivatives, and/or salts of these compounds.

Furthermore, it is advantageous in accordance with the invention, when one or more preservatives are present in a concentration from 0.01 to 3% by weight, preferably with a concentration from 0.1 to 2% by weight, each based on the total weight of the preparation.

Preferably, besides one or more water phases, the cleansing preparation of the invention can additionally comprise one or more oil phases. These are present, for example, in the form of W/O-, O/W-, W/O/W-, or O/W/o emulsions. Such formulations may preferably also be a microemulsion (e.g. a PIT emulsion).

According to the invention, the cleansing preparations of the invention may contain besides the foregoing substances, additives as are customary in cosmetics, for example, perfume, dyes, antimicrobial agents, refatting agents, complexing and sequestering agents, plant extracts, vitamins, active agents, preservatives, bactericides, UV light protection filters, repellents, self-tanners, depigmentors, pigments, which have a coloring effect, softening, moisturizing, and/or humectant substances, or other common constituents of a cosmetic or dermatological formulation, such as emulsifiers, polymers, foam stabilizers, electrolytes, organic solvents, or silicone derivatives.

In accordance with the invention, the cleansing preparation of the invention may advantageously contain as aqueous solution or aqueous phase besides water also other ingredients, for example, alcohols, diols or polyols of a low carbon number, as well as ethers thereof, preferably ethanol, isopropanol, propylene glycol, glycerol, ethylene glycol, ethylene glycol monoethyl- or -monobutyl ether, propylene glycol monomethyl, -monoethyl-, or -monobutyl ether, polypropylene glycol monoethyl, diethylene glycol monomethyl or -monoethyl ether, and analogous products, furthermore alcohols of a low carbon number, for example, ethanol, isopropanol, 1,2-propanediol, and glycerol.

Advantageous moisturizing or humectant agents (so-called moisturizers) for the purposes of the present invention are, for example, glycerin, lactic acid and/or lactates, in particular, sodium lactate, butylene glycol, propylene glycol, biosaccharide gum-1, glycine soya, ethylhexyloxyglycerin, pyrrolidone carboxylic acid, and urea. Furthermore, it is of special advantage to use polymeric moisturizers from the group of the water-soluble and/or water-swellable and/or water-gellable polysaccarides. Particularly advantageous are, for example, hyaluronic acid, chitosan, and/or a fucose-rich polysaccharide, which is filed in the Chemical Abstracts under the Registry Number 178463-23-5, and which can be obtained, for example, under the name Fucogel® 1000 from SOLABIA S.A.

In accordance with the invention, it is advantageous to add film formers (conditioners) to the cleansing preparations of the invention.

Advantageous water-soluble or dispersible film formers are, for example, polyurethane (for example, the Avalure® types from Goodrich), dimethicone copolyol polyacrylate (Silsoft Surface® from Witco Organo Silicones Group) PVP/VA (VA=vinyl acetate) copolymer (Luvisol VA 64 Powder from BASF), etc.

Advantageous fat-soluble film formers are, for example, the film formers from the group of the polymers on the basis of polyvinyl pyrrolidone (PVP)

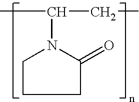

Advantageous are the copolymers of polyvinyl pyrrolidone, for example, the PVP of hexadecene copolymer, and the PVP eicosen copolymer, which are available under the tradenames Antaron V216 and Antaron V220 from GAF Chemicals Corporation, as well as tricontayl PVP, and the like.

Suitable as film formers with at least one partially quaternized nitrogen group are preferably those which are selected from the group of the substances that bear the name “polyquaternium” in accordance with the INCI Nomenclature (International Nomenclature Cosmetic Ingredient), for example:

| | |
|---|---|
| Polyquaternium-2 | (Chemical Abstracts No. 63451-27-4 for example, |
| | Mirapol ® A-15) |
| Polyquaternium-5 | (Copolymers from acrylamide, β-methacryloxy- |
| | ethyltriethyl ammoniummethosulfate, CAS No. |
| | 26006-22-4) |
| Polyquaternium-6 | Homopolymer of N,N-dimethyl-N-2-propenyl-2- |
| | propen-1-aminium chloride, CAS No. 26062-79-3, |
| | for example, Merquat ® 100 |
| Polyquaternium-7 | N,N-Dimethyl-N-2-propenyl-2-propen aminium |
| | chloride, polymers with 2-propenamide, CAS No. |
| | 26590-05-6, for example, Merquat ® S |
| Polyquaternium-10 | Quaternary ammonium salt of hydroxyethyl |
| | cellulose, CAS Nos. 53568-66-4, 55353-19-0, |
| | 54351-50-7, 68610-92-4, 81859-24-7, for |
| | example, Celquat ® SC-230M |
| Polyquaternium-11 | Vinylpyrrolidone/dimethyl aminoethyl- |
| | methacrylate copolymer/diethylsulfate |
| | reaction product, CAS No. |
| | 53633-54-8, for example, Gafquat ®755N |
| Polyquaternium-16 | Vinylpyrrolidone/vinylimidazolinium |
| | methochloride copolymer, CAS No. 29297-55-0, |
| | for example, Luviquat ® HM552 |
| Polyquaternium-17 | CAS No. 90624-75-2, for example, |
| | Mirapol ®AD-1 |
| Polyquaternium-19 | Quaternized water-soluble polyvinyl alcohol |
| Polyquaternium-20 | Water-dispersible, quaternized polyvinyl |
| | octadecyl ether |
| Polyquaternium-21 | Polysiloxane-polydimethyl-dimethylammonium |
| | acetate copolymers for example, Abil ® |
| | B 9905 |
| Polyquaternium-22 | Dimethyldiallyl ammonium chloride/acrylic acid |
| | copolymer, CAS No. 53694-7-0, for example, |
| | Merquat ®280 |
| Polyquaternium-24 | Polymeric quaternary ammonium salt of the |
| | hydroxyethyl cellulose, reaction product with |
| | a lauryl dimethyl ammonium-substituted epoxide, |
| | CAS No. 107987-23-5, for example, Quatrisoft ® |
| | LM-200 |
| Polyquaternium-28 | Vinylpyrrolidone/methacryl-amidopropyl-trimethyl |
| | ammonium chloride copolymer, for example, |
| | Gafquat ®HS-100 |
| Polyquaternium-29 | for example, Lexquat ® CH |
| Polyquaternium-31 | CAS No. 136505-02-7, for example, Hypan ® |
| | QT 100 |
| Polyquaternium-32 | N,N,N-trimethyl-2-[(2-methyl-1-oxo-2- |
| | propenyl)oxy]-ethanaminium chloride, polymer |
| | with 2-propenamide, CAS No. 35429-19-7 |
| Polyquaternium-37 | CAS No. 26161-33-1 |
| Polyquaternium-44 | Copolymeric quaternary ammonium salt consisting |
| | of vinylpyrrolidone and quaternized imidazoline, |
| | for example, Luviquat Care ® |

Preferred and advantageous are the polymers Polyquaternium-10, Polyquaternium-22, and Polyquaternium-44.

A particularly preferred film former in accordance with the invention is Polyquaternium-10 (Ucare Polymer JR-125®, Ucare Polymer JR-400® from Amerchol).

Further advantageous film formers are quaternized guar gum derivatives, such as, for example, guar hydroxypropyl trimonium chloride (Jaguar Excel@, Jaguar C 162® from Rhodia), and/or cellulose derivatives.

The oil phase of the cleansing preparations according to the invention is advantageously chosen from the group of polar oils, for example from the group of lecithins and of fatty acid triglycerides, namely the triglycerol esters of saturated and/or unsaturated, branched and/or unbranched alkanecarboxylic acids of a chain length from 8 to 24, in particular 12 to 18 carbon atoms. The fatty acid triglycerides can, for example, advantageously be chosen from the group of synthetic, semi-synthetic and natural oils, such as, for example, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil, and the like.

Further advantageous polar oil components can also be chosen for the purposes of the present invention from the group of esters of saturated and/or unsaturated, branched and/or unbranched alkanecarboxylic acids of a chain length from 3 to 30 carbon atoms and saturated and/or unsaturated, branched and/or unbranched alcohols of a chain length from 3 to 30 carbon atoms, and from the group of esters of aromatic carboxylic acids and saturated and/or unsaturated, branched and/or unbranched alcohols of a chain length from 3 to 30 carbon atoms. Such ester oils can then advantageously be chosen from the group consisting of octyl palmitate, octyl cocoate, octyl isostearate, octyl dodecyl myristate, octyldodecanol, cetearyl isononoanoate, isopropyl myristate, isopropyl palmitate, isopropyl stearate, isopropyl oleate, n-butyl stearate, n-hexyl laurate, n-decyl oleate, isocotyl stearate, isnonyl stearate, isononyl isononanoate, 2-ethylhexyl palmitate, 2-ethyhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase can advantageously be chosen from the group of dialkyl ethers and dialkyl carbonates, for example dicaprylyl ether (Cetiol OE) and/or dicaprylyl carbonate, for example, that available under the trade name Cetiol CC from Cognis It is also preferred to choose the oil component or components from the group consisting of isoeicosane, neopentyl glycol diheptanoate, propylene glycol dicaprylate/dicaprate, caprylic/capric/diglyceride succinate, butylene glycol dicaprylate/dicaprate, cocoglycerides (e.g. Myritol® 331 from Henkel), C₁₂₋₁₃ alkyl lactate, di-C₁₂₋₁₃ alkyl tartrate, triisostearin, dipentaerythrityl hexcaprylate/hexacaprate, propylene glycol monoiostearate, tricaprylin, dimethyl isosorbide. It is particularly advantageous, when the oil phase of the formulations according to the invention has a content of C₁₂₋₁₅-alkyl benzoate or consists entirely of this.

Advantageous oil components are also, for example, butyloctyl salicylate (for example, that available under the trade name Hallbrite BHB from CP Hall), hexadecyl benzoate and butyloctyl benzoate and mixtures thereof (Hallstar AB) and/or diethylhexyl naphthalate (Corapan® TQ from Haarmann & Reimer).

In accordance with the invention, it is also advantageous to use as oil component paraffinum liquidum.

In accordance with the invention, advantageous embodiments of the substrates according to the invention are characterized in that the cleansing preparation contains skin-caring oils and/or emulsions. According to the invention, the cleansing preparation preferably contains skin-caring oils and/or emulsions in concentrations smaller than 0.5% by weight.

The invention also relates to a method of producing substrates. This method is characterized in that the cosmetic preparation is applied to the substrate at a temperature of 40° C. or higher than 40° C., and that it is subsequently cooled. In accordance with the invention, it is preferred to cool the preparation that has been applied to the substrate, to room temperature. In this connection, temperatures from 15° C. to 25° C. are especially preferred.

The cooling of the heated preparation causes fatty acid salts to separate (or crystallize) on and/or in the substrate. The further constituents of the preparation according to the invention are set on the substrate by being included in the separated fatty acid salts, and/or by adhering thereto. Accordingly, the method for impregnating and/or setting cosmetic preparations on substrates is also characterized in that fatty acid salts dissolved in a heated cosmetic preparation are applied to the substrate, and that they are separated on the substrate by subsequent cooling.

In accordance with the invention, it is possible and advantageous to apply the preparations of the invention to the substrates by the following methods:

a) Direct coating
The impregnation solution is continuously supplied to a scraper arrangement (scraper bar, counterrotating roller system, or the like), and transferred by contact to a moving substrate.

b) Spray coating
The liquid impregnation solution is sprayed onto the moving substrate.

c) Dip coating
The fabric material passes through a bath filled with the liquid impregnation solution, or an entire roll of fabric is immersed into the impregnation solution.

In accordance with the invention the method for producing the substrates of the invention is also characterized in that the impregnated substrate is dried after cooling.

In accordance with the invention, the substrates of the invention are used for cleansing and for the care of the body and face.

In accordance with the invention, the substrates of the invention are used for cleansing and for the care of the skin.

In accordance with the invention, the substrates are used for cleansing facial skin by removing decorative cosmetics (the so-called “makeup removal”), as well as for cleansing unclean skin.

Last but not least, it is in accordance with the invention to use the substrates of the invention for cleansing and for the care of skin appendages, in particular hair and nails.

It is likewise in accordance with the invention to use the wipe of the invention as a mild, foaming cleansing utensil.

The following examples are intended to illustrate the present invention without limiting it. Unless otherwise specified, all quantity, constituent, and percentage values are based on the weight and total quantity or total weight of the preparations.

| | 1 | 2 | 3 | 4 | 5 |
|---|---|---|---|---|---|
| Lauric acid | 2 | 2 | — | 2 | 2 |
| Myristic acid | 1 | — | 2 | 2 | 3 |
| Palmitic acid | 1 | 2 | 2 | 2 | — |
| Stearic acid | 2 | 1 | 2 | 1 | 2 |
| Potassium hydroxide | 4 | 3 | 3 | 4 | 4 |
| Sodium laurylether sulfate | 3 | 3 | 4 | 2 | — |
| Decylpolyglucoside | 2 | 1 | — | 1 | 2 |
| Sodium methylcocoyl taurate | 1 | — | — | — | 2 |
| Polyoxyethylene(20)sorbitanmonolaurate | — | 2 | 1 | — | — |
| Sodium cocoyl glutamate | — | — | 2 | 3 | 3 |
| Polyquaternium-10 | 0.1 | 0.2 | 0.1 | 0.1 | 0.1 |
| Propyleneglycol | 1 | 0.5 | — | — | 1 |
| Glycerin | — | — | — | 1 | 1 |
| Etidronic acid | 1 | 1 | 1 | 0.5 | 0.5 |
| Potassium chloride | 2 | 2 | 2 | 2 | 2 |
| Phenoxyethanol | 0.5 | 0.5 | 0.6 | 0.5 | 0.5 |
| Perfume | 1 | 1 | 1 | 1 | 1 |
| Water | ad 100 | ad 100 | ad 100 | ad 100 | ad 100 |

1. Substrate impregnated with a cosmetic preparation containing one or more fatty acid salts.

2. Substrate of claim 1, characterized in that the substrate is a wipe of a nonwoven fabric.

3. Substrate of one of claims 1 or 2, characterized in that the fatty acid salt or salts are selected from the group of fatty acids, which comprise 8 to 20 carbon atoms per molecule.

4. Substrate of one of claims 1-3, characterized in that the fatty acid salt or salts are selected from the group of potassium laurate, potassium myristate, potassium palmitate, potassium stearate, and potassium oleate.

5. Substrate of one of claims 1-4, characterized in that the cosmetic preparation contains at the time of its application to the substrate one or more fatty acid salts in a concentration from 0.1 to 20% by weight based on the total weight of the preparation.

6. Substrate of one of claims 1-5, characterized in that the cosmetic preparation contains one or more surfactants.

7. Method of impregnating and/or setting cosmetic preparations on substrates, characterized in that fatty acid salts dissolved in a heated cosmetic preparation are applied to the substrate, and separated on the substrate by subsequent cooling.

8. Method of producing substrates of one of claims 1-7, characterized in that the cosmetic preparation is applied to the substrate at a temperature of 40° C. or greater than 40° C., and that it is subsequently cooled.

9. Method of producing substrates according to claim 8, characterized in that the impregnated substrate is dried after cooling.

10. Use of a substrate of one of the foregoing claims for cleansing and the care of body and face.
